(19) United States
(12) Patent Application Publication
(10) Pub. No.: US 2021/0330901 A1
(43) Pub. Date: Oct. 28, 2021

(54) Systems and Methods to Administer Pharmaceuticals

(71) Applicant: Douglas G. Metcalf, Boulder, CO (US)

(72) Inventor: Douglas G. Metcalf, Boulder, CO (US)

(21) Appl. No.: 17/278,305

(22) PCT Filed: Sep. 23, 2019
(86) PCT No.: PCT/US19/52470

§ 371 Date: Mar. 20, 2021

Related U.S. Application Data
(60) Provisional application No. 62/735,148, filed on Sep. 23, 2018.

(51) Int. Cl. A61M 11/04; A61K 9/00; A61K 31/05; A23L 2/52; A23L 33/105; A23D 7/005; G16H 40/63; G16H 20/10; G06F 21/32; G05B 19/4155; H05B 1/02

(52) U.S. Cl. CPC A61M 11/042; G05B 2219/50333; A61K 31/05; A23L 2/52; A23L 33/105; A23D 7/0053; G16H 40/63; G16H 20/10; G06F 21/32; G05B 19/4155; H05B 1/025; A61M 2205/0233; A61M 2205/50; A61M 2205/609; A61M 2205/276; A23V 2002/00; A61K 9/0095

(57) ABSTRACT

Various aspects of this disclosure relate to containers, devices, and methods to administer single doses of pharmaceutical agents.

### CROSS REFERENCE TO RELATED APPLICATIONS

This patent is the U.S. National Phase under 35 U.S.C. 371 of International Application No. PCT/US2019/052470, which claims priority to U.S. Provisional Patent Application No. 62/735,148, filed Sep. 23, 2018, both of which are incorporated by reference in their entirety.

### BACKGROUND

Numerous hydrophobic pharmaceutical agents fail pre-clinical and clinical trials due to limited bioavailability. Vaporization presents a widely-unexplored alternative to administer pharmaceuticals that lack robust bioavailability by other routes of administration. Vaporization is technically challenging because accurate dosing is difficult to achieve and because vaporization may result in the thermal degradation of a pharmaceutical agent. Excipients also often cause undesirable side effects when heated, and a number of deaths have been recently linked to vape oil products. Improved methods and devices to vaporize pharmaceuticals could result in the further development of multitudes of abandoned drug candidates.

### SUMMARY

Some aspects of the disclosure relate to a container comprising (i) a hermetically-sealed chamber, (ii) a composition contained within the hermetically-sealed chamber, and (iii) a thermally-conductive surface in thermal communication with the composition, wherein the thermally-conductive surface has a thermal conductivity of 5 to 10,000 watts per meter-Kelvin; the composition comprises 0.1 to 20 micromoles of a pharmaceutical agent; the hermetically-sealed chamber has an initial volume of 0.01 to 50 microliters when the composition is not in a gas phase; and the hermetically-sealed chamber has an inflated volume of 0.05 to 5 milliliters when the composition is in a gas phase. In some embodiments, a composition comprises a pharmaceutical agent selected from one, two, three, four, five, six, seven, or each of cannabidiol, cannabidiolic acid, cannabivarin, cannabivarin carboxylic acid, tetrahydrocannabinol, tetrahydrocannabinolic acid, tetrahydrocannabivarin, and tetrahydrocannabivarin carboxylic acid. In some specific embodiments, a composition comprises (i) a pharmaceutical agent selected from one, two, three, four, five, six, seven, or each of cannabidiol, cannabidiolic acid, cannabivarin, cannabivarin carboxylic acid, tetrahydrocannabinol, tetrahydrocannabinolic acid, tetrahydrocannabivarin, tetrahydrocannabivarin carboxylic acid; and (ii) a vehicle selected from one or more of alpha-bisabolol, alpha-pinene, beta-caryophyllene, beta-pinene, humulene, limonene, linalool, myrcene, nerolidol, terpineol, terpinolene, water, and ethanol. In some very specific embodiments, a composition comprises a pharmaceutical agent selected from one or both of cannabidiol and tetrahydrocannabinol; and a vehicle selected from one or both of water and ethanol.

### BRIEF DESCRIPTION OF THE DRAWINGS

FIG. 1A is a diagram of a container comprising (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase.

FIG. 1B is a diagram of a container comprising (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is in a gas phase.

FIG. 2A is a diagram of a container comprising (i) a hermetically-sealed chamber 1, (ii) a composition comprising both a pharmaceutical agent 2 and a vehicle 4 contained within the hermetically-sealed chamber 1, and (iii) a thermally-conductive surface 3 in thermal communication with the composition, wherein neither the pharmaceutical agent 2 nor the vehicle 4 are in a gas phase.

FIG. 2B is a diagram of a container comprising (i) a hermetically-sealed chamber 1, (ii) a composition comprising both a pharmaceutical agent 2 and a vehicle 4 contained within the hermetically-sealed chamber 1, and (iii) a thermally-conductive surface 3 in thermal communication with the composition, wherein the pharmaceutical agent 2 and the vehicle 4 are in a gas phase.

FIG. 3A is a diagram of a device comprising a heating compartment 5 and a heating element 6, wherein the heating compartment 5 is in receipt of a container. The container comprises (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase. The heating element 6 is in thermal communication with the thermally-conductive surface 3 such that the heating element 6 is operable to both heat the thermally-conductive surface 3 and heat the composition to vaporize at least a portion of the pharmaceutical agent 2.

FIG. 3B is a diagram of a device comprising a heating compartment 5 and a heating element 6, wherein the heating compartment 5 is in receipt of a container. The container comprises (i) a hermetically-sealed chamber 1, (ii) a composition comprising a vaporized pharmaceutical agent 2 contained within the hermetically-sealed chamber 1, and (iii) a thermally-conductive surface 3 in thermal communication with the composition. The heating element 6 is in thermal communication with the thermally-conductive surface 3, and the heating element 6 has heated the thermally-conductive surface 3 and the composition to produce the vaporized pharmaceutical agent 2.

FIG. 3C is a diagram of a device comprising a heating compartment 5 and a heating element 6, wherein the heating compartment 5 is in receipt of a container. The container comprises an unsealed chamber 1 and a thermally-conductive surface 3. The heating element 6 is in thermal communication with the thermally-conductive surface 3, and the heating element 6 has heated the thermally-conductive surface 3 and a composition previously contained within the unsealed chamber 1 to produce a vaporized pharmaceutical agent 2.

FIG. 4A is a diagram of a device comprising a heating compartment 5, a heating element 6, an administration path 7 in fluid communication with the heating compartment 5, and an actuator 8 in mechanical communication with the heating compartment 5, wherein the heating compartment 5 is in receipt of a container. The container comprises (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase. The heating element 6 is in thermal communication with the thermally-conductive surface 3 such that the heating element 6 is operable to both heat the thermally-conductive surface 3 and heat the composition to vaporize at least a portion of the pharmaceutical agent 2.

FIG. 4B is a diagram of a device comprising a heating compartment 5, a heating element 6, an administration path 7 in fluid communication with the heating compartment 5, and an actuator 8 in mechanical communication with the heating compartment 5, wherein the heating compartment 5 is in receipt of a container. The container comprises (i) a hermetically-sealed chamber 1, (ii) a composition comprising a vaporized pharmaceutical agent 2 contained within the hermetically-sealed chamber 1, and (iii) a thermally-conductive surface 3 in thermal communication with the composition. The heating element 6 is in thermal communication with the thermally-conductive surface 3, and the heating element 6 has heated the thermally-conductive surface 3 and the composition to produce the vaporized pharmaceutical agent 2.

FIG. 4C is a diagram of a device comprising a heating compartment 5, a heating element 6, an administration path 7 in fluid communication with the heating compartment 5, and an actuator 8 in mechanical communication with the heating compartment 5, wherein the heating compartment 5 is in receipt of a container. The container comprises an unsealed chamber 1 and a thermally-conductive surface 3. The heating element 6 is in thermal communication with the thermally-conductive surface 3, and the heating element 6 has heated the thermally-conductive surface 3 and a composition previously contained within the unsealed chamber 1 to produce a vaporized pharmaceutical agent 2. The unsealed chamber 1 is connected to the administration path 7, and the actuator 8 has propelled the vaporized pharmaceutical agent 2 from the unsealed chamber 1 and into the administration path 7.

FIG. 5A is a diagram of a device comprising a heating compartment 5, a heating element 6, and a controller 9, wherein the heating compartment 5 is in receipt of a container. The container comprises (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase. The controller 9 is in directive communication with the heating element 6 such that the controller 9 is operable to run a heating program that causes the heating element 6 to heat. The heating element 6 is in thermal communication with the thermally-conductive surface 3 such that the heating element 6 is operable to both heat the thermally-conductive surface 3 and heat the composition to vaporize at least a portion of the pharmaceutical agent 2.

FIG. 5B is a diagram of a device comprising a heating compartment 5, a heating element 6, and a controller 9, wherein the heating compartment 5 is in receipt of a container. The container comprises (i) a hermetically-sealed chamber 1, (ii) a composition comprising a vaporized pharmaceutical agent 2 contained within the hermetically-sealed chamber 1, and (iii) a thermally-conductive surface 3 in thermal communication with the composition. The controller 9 is in directive communication with the heating element 6, and the controller 9 has run a heating program that caused the heating element 6 to heat. The heating element 6 is in thermal communication with the thermally-conductive surface 3, and the heating element 6 has heated the thermally-conductive surface 3 and the composition to produce the vaporized pharmaceutical agent 2.

FIG. 5C is a diagram of a device comprising a heating compartment 5, a heating element 6, and a controller 9, wherein the heating compartment 5 is in receipt of a container. The container comprises an unsealed chamber 1 and a thermally-conductive surface 3. The controller 9 is in directive communication with the heating element 6, and the controller 9 has run a heating program that caused the heating element 6 to heat. The heating element 6 is in thermal communication with the thermally-conductive surface 3, and the heating element 6 has heated the thermally-conductive surface 3 and a composition previously contained within the unsealed chamber 1 to produce a vaporized pharmaceutical agent 2.

FIG. 6A is a diagram of a device comprising a heating compartment 5, a heating element 6, an administration path 7 in fluid communication with the heating compartment 5, an actuator 8 in mechanical communication with the heating compartment 5, and a controller 9, wherein the heating compartment 5 is in receipt of a container. The container comprises (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase. The controller 9 is in directive communication with the heating element 6 such that the controller 9 is operable to run a heating program that causes the heating element 6 to heat. The heating element 6 is in thermal communication with the thermally-conductive surface 3 such that the heating element 6 is operable to both heat the thermally-conductive surface 3 and heat the composition to vaporize at least a portion of the pharmaceutical agent 2.

FIG. 6B is a diagram of a device comprising a heating compartment 5, a heating element 6, an administration path 7 in fluid communication with the heating compartment 5, an actuator 8 in mechanical communication with the heating compartment 5, and a controller 9, wherein the heating compartment 5 is in receipt of a container. The container comprises (i) a hermetically-sealed chamber 1, (ii) a composition comprising a vaporized pharmaceutical agent 2 contained within the hermetically-sealed chamber 1, and (iii) a thermally-conductive surface 3 in thermal communication with the composition. The controller 9 is in directive communication with the heating element 6, and the controller 9 has run a heating program that caused the heating element 6 to heat. The heating element 6 is in thermal communication with the thermally-conductive surface 3, and the heating element 6 has heated the thermally-conductive surface 3 and the composition to produce the vaporized pharmaceutical agent 2.

FIG. 6C is a diagram of a device comprising a heating compartment 5, a heating element 6, an administration path 7 in fluid communication with the heating compartment 5, an actuator 8 in mechanical communication with the heating compartment 5, and a controller 9, wherein the heating compartment 5 is in receipt of a container. The container comprises an unsealed chamber 1 and a thermally-conductive surface 3. The controller 9 is in directive communication with the heating element 6, and the controller 9 has run a heating program that caused the heating element 6 to heat. The heating element 6 is in thermal communication with the thermally-conductive surface 3, and the heating element 6 has heated the thermally-conductive surface 3 and a composition previously contained within the unsealed chamber 1 to produce a vaporized pharmaceutical agent 2. The unsealed chamber 1 is connected to the administration path 7, and the actuator 8 has propelled the vaporized pharmaceutical agent 2 from the unsealed chamber 1 and into the administration path 7.

FIG. 7A is a diagram of a device comprising a heating compartment 5, a heating element 6, an administration path 7 in fluid communication with the heating compartment 5, an actuator 8 in mechanical communication with the heating compartment 5, a controller 9, a battery 10, and an interface 11, wherein the heating compartment 5 is in receipt of a container. The container comprises (i) a hermetically-sealed chamber 1, (ii) a composition comprising a pharmaceutical agent 2 contained within the hermetically-sealed chamber 1, and (iii) a thermally-conductive surface 3 in thermal communication with the composition, wherein the pharmaceutical agent 2 is not in a gas phase. The controller 9 is in electronic communication with the interface 11 such that the controller 9 is operable to receive at least one input from the interface 11 to run an authorization program to determine whether the controller 9 is authorized to run a heating program based on one or more inputs that include the at least one input. The controller 9 is in directive communication with the heating element 6 such that the controller 9 is operable to run a heating program that causes the heating element 6 to heat. The heating element 6 is in electrical communication with the battery 10, such that the battery 10 is operable to power the heating element 6. The heating element 6 is in thermal communication with the thermally-conductive surface 3 such that the heating element 6 is operable to both heat the thermally-conductive surface 3 and heat the composition to vaporize at least a portion of the pharmaceutical agent 2.

FIG. 7B is a diagram of a device comprising a heating compartment 5, a heating element 6, an administration path 7 in fluid communication with the heating compartment 5, an actuator 8 in mechanical communication with the heating compartment 5, a controller 9, a battery 10, and an interface 11, wherein the heating compartment 5 is in receipt of a container. The container comprises (i) a hermetically-sealed chamber 1, (ii) a composition comprising a vaporized pharmaceutical agent 2 contained within the hermetically-sealed chamber 1, and (iii) a thermally-conductive surface 3 in thermal communication with the composition. The controller 9 is in electronic communication with the interface 11 such that the controller 9 is operable to receive at least one input from the interface 11 to run an authorization program to determine whether the controller 9 is authorized to run a heating program based on one or more inputs that include the at least one input. The controller 9 is in directive communication with the heating element 6, and the controller 9 has run a heating program that caused the heating element 6 to heat. The heating element 6 is in electrical communication with the battery 10, such that the battery 10 is operable to power the heating element 6. The heating element 6 is in thermal communication with the thermally-conductive surface 3, and the heating element 6 has heated the thermally-conductive surface 3 and the composition to produce the vaporized pharmaceutical agent 2.

FIG. 7C is a diagram of a device comprising a heating compartment 5, a heating element 6, an administration path 7 in fluid communication with the heating compartment 5, an actuator 8 in mechanical communication with the heating compartment 5, a controller 9, a battery 10, and an interface 11, wherein the heating compartment 5 is in receipt of a container. The container comprises an unsealed chamber 1 and a thermally-conductive surface 3. The controller 9 is in electronic communication with the interface 11 such that the controller 9 is operable to receive at least one input from the interface 11 to run an authorization program to determine whether the controller 9 is authorized to run a heating program based on one or more inputs that include the at least one input. The controller 9 is in directive communication with the heating element 6, and the controller 9 has run a heating program that caused the heating element 6 to heat. The heating element 6 is in electrical communication with the battery 10, such that the battery 10 is operable to power the heating element 6. The heating element 6 is in thermal communication with the thermally-conductive surface 3, and the heating element 6 has heated the thermally-conductive surface 3 and a composition previously contained within the unsealed chamber 1 to produce a vaporized pharmaceutical agent 2. The unsealed chamber 1 is connected to the administration path 7, and the actuator 8 has propelled the vaporized pharmaceutical agent 2 from the unsealed chamber 1 and into the administration path 7.

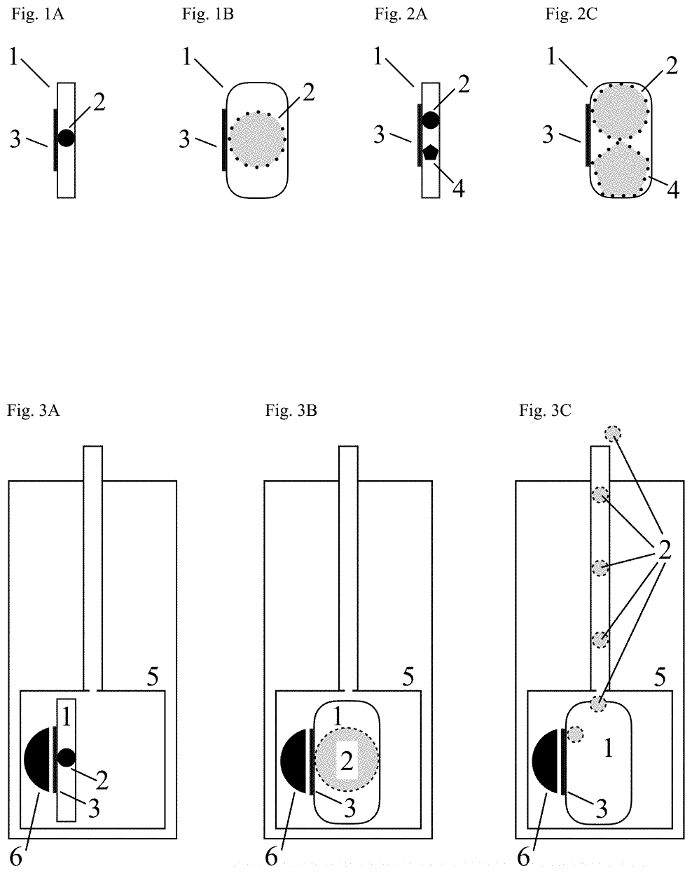

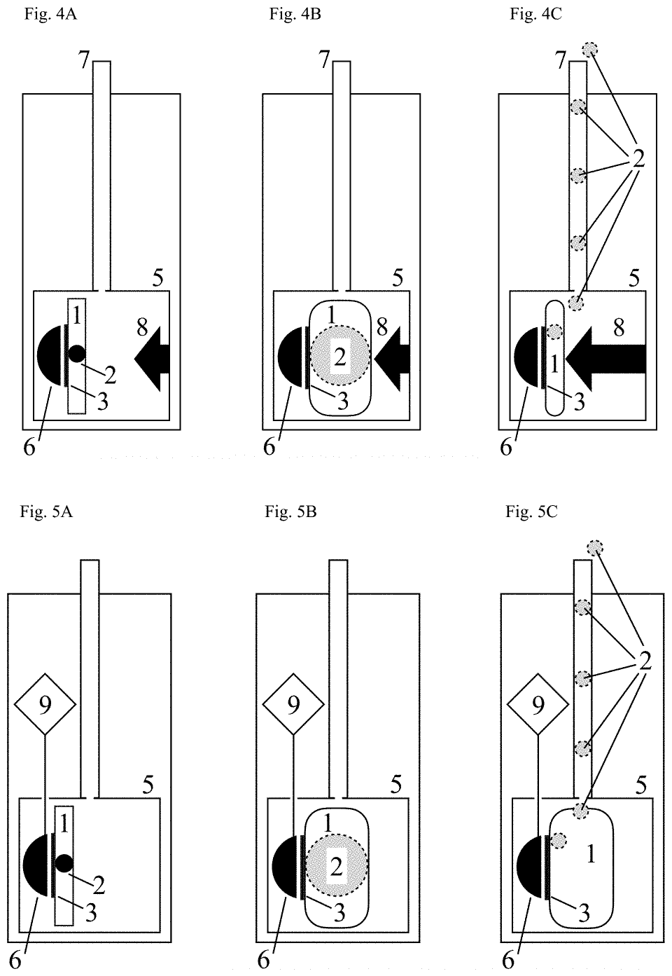

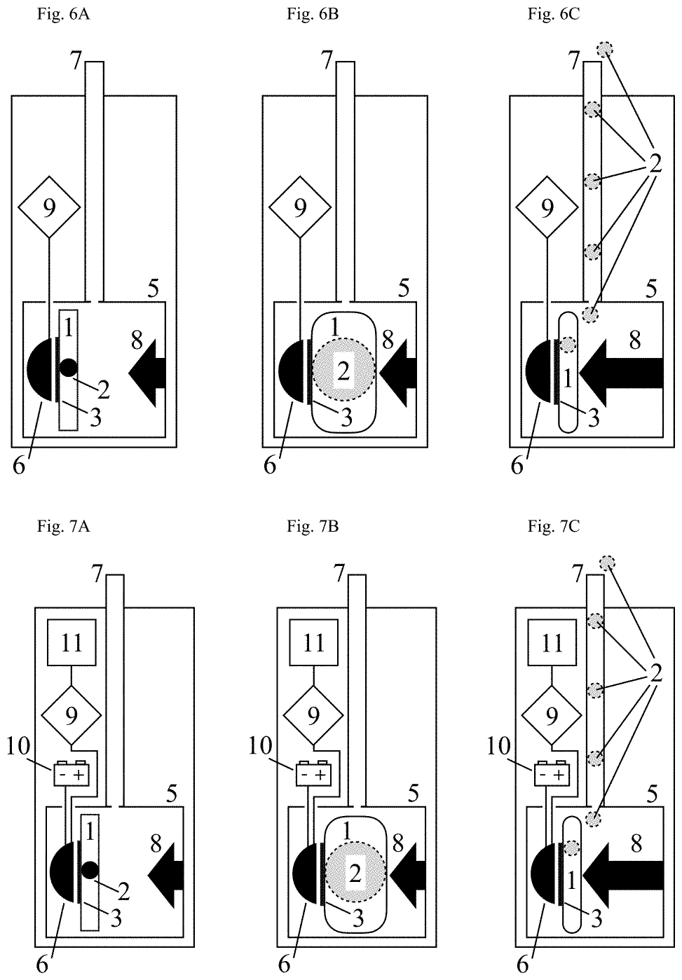

### DETAILED DESCRIPTION

Various aspects of the disclosure relate to a container. Some of the containers of the disclosure are designed to contain a single dose of a pharmaceutical agent to be administered by inhalation after the single dose of the pharmaceutical agent is vaporized.

In some embodiments, a container comprises (i) a hermetically-sealed chamber, (ii) a composition contained within the hermetically-sealed chamber, and (iii) a thermally-conductive surface in thermal communication with the composition.

In some embodiments, a composition comprises 0.01 to 500 micromoles of a pharmaceutical agent. In some specific embodiments, a composition comprises 0.01 to 10, 0.1 to 20, 0.5 to 5, or 10 to 500 micromoles of a pharmaceutical agent. In some very specific embodiments, a composition comprises 0.1 to 20 micromoles of a pharmaceutical agent selected from one or more of cannabidiol, cannabidiolic acid, cannabivarin, cannabivarin carboxylic acid, tetrahydrocannabinol, tetrahydrocannabinolic acid, tetrahydrocannabivarin, and tetrahydrocannabivarin carboxylic acid.

In some embodiments, a pharmaceutical agent has a boiling point between 26 and 260 degrees Celsius. The term “boiling point” refers to boiling points at atmospheric pressure. In some specific embodiments, a pharmaceutical agent is selected from one or more of acetylsalicylic acid, alpha-bisabolol, alpha-pinene, amphetamine, beta-caryophyllene, beta-pinene, cannabidiol, cannabidiolic acid, cannabivarin, cannabivarin carboxylic acid, chlormethine, cocaine, diethyl ether, ethanol, humulene, ibuprofen, limonene, linalool, mescaline, methamphetamine, 3,4-methylenedioxymethamphetamine, methylphenidate, morphine, myrcene, nerolidol, nicotine, N,N-dimethyltryptamine, phencyclidine, phenelzine, phentermine, safrole, terpineol, terpinolene, tetrahydrocannabinol, tetrahydrocannabinolic acid, tetrahydrocannabivarin, and tetrahydrocannabivarin carboxylic acid.

In some embodiments, a thermally-conductive surface has a thermal conductivity greater than 2 watts per meter-Kelvin (W·m⁻¹·K⁻¹). The term “thermal conductivity” refers to thermal conductivity at atmospheric pressure and 20 degrees Celsius. In some specific embodiments, a thermally-conductive surface has a thermal conductivity of 7 to 1000 watts per meter-Kelvin. In some very specific embodiments, a thermally-conductive surface has a thermal conductivity of 50 to 500 watts per meter-Kelvin.

In some embodiments, a container comprises a metal, and the metal has a thermal conductivity of at least 2 watts per meter-Kelvin. In some specific embodiments, a container comprises a metal, and the metal has a thermal conductivity of 7 to 1000 watts per meter-Kelvin.

In some embodiments, a hermetically-sealed chamber contains less than 1 milligram of molecular oxygen (O₂). In some specific embodiments, a hermetically-sealed chamber contains less than 0.1 milligrams of molecular oxygen. In some very specific embodiments, a hermetically-sealed chamber contains less than 0.01 milligrams of molecular oxygen.

In some embodiments, a thermally-conductive surface has a surface area of less than 0.01 meters squared. In some specific embodiments, a thermally-conductive surface has a surface area of 0.00001 to 0.01 meters squared.

In some embodiments, 0.000001 to 0.01 meters squared of a thermally-conductive surface is in physical communication with 0.000001 to 0.01 meters squared of a composition. In some specific embodiments, 0.00001 to 0.01 meters squared of a thermally-conductive surface is in physical communication with 0.00001 to 0.01 meters squared of a composition.

In some embodiments, a hermetically-sealed chamber comprises a hermetic layer. A hermetic layer inhibits the diffusion of gases. In some specific embodiments, a thermally-conducive surface is permeable to gases, and a hermetic layer covers the thermally-conductive surface to inhibit the diffusion of gases into or out of a hermetically-sealed chamber. In some specific embodiments, a hermetic layer comprises a polymer. In some specific embodiments, a hermetic layer is 1 to 500 micrometers thick.

In some embodiments, a container comprises an exit path, wherein the container is configured to inhibit the flow of a composition through the exit path until after a pharmaceutical agent of the composition is heated to a temperature above the boiling point of the pharmaceutical agent.

In some embodiments, a container lacks a heating element. In some embodiments, a container lacks a battery. In some specific embodiments, a container lacks both a heating element and a battery.

In some embodiments, a composition comprises 0.01 to 10 micromoles of a second pharmaceutical agent; the second pharmaceutical agent has a different molecular formula than the pharmaceutical agent; the pharmaceutical agent is neither a thermal decomposition product nor an oxidation product of the second pharmaceutical agent; the second pharmaceutical agent is neither a thermal decomposition product nor an oxidation product of the pharmaceutical agent; and the second pharmaceutical agent has a boiling point between 26 and 260 degrees Celsius. In some embodiments, a pharmaceutical agent is tetrahydrocannabinol, and a second pharmaceutical agent is cannabidiol. In some embodiments, a pharmaceutical agent is cannabidiol, and a second pharmaceutical agent is tetrahydrocannabinol. In some embodiments, a pharmaceutical agent is tetrahydrocannabinol, and a second pharmaceutical agent is tetrahydrocannabivarin.

In some embodiments, a hermetically-sealed chamber is configured such that a composition contained within the hermetically-sealed container can exist in either a liquid phase or a gas phase within the hermetically-sealed chamber; the hermetically-sealed chamber has an initial volume when the composition is in the liquid phase; the hermetically-sealed chamber has an inflated volume when the composition is in the gas phase; and the inflated volume is at least 900% greater than the initial volume.

In some embodiments, a composition is not a gas. In some specific embodiments, a composition is a liquid. In some embodiments, a composition is a gas.

In some embodiments, a composition comprises a vehicle. A vehicle consists of molecules that vaporize by heating to increase the volume of a composition comprising a vaporized pharmaceutical agent. A composition comprising a pharmaceutical agent and a vehicle typically comprises the vehicle at a greater amount by mole than the pharmaceutical agent.

In some embodiments, a composition comprises a pharmaceutical agent and a vehicle, and each molecule of the vehicle has a different molecular formula than the pharmaceutical agent.

In some embodiments, a composition comprises 1 to 10,000 micromoles of a vehicle. In some specific embodiments, a composition comprises 1 to 1,000 micromoles of a vehicle. In some very specific embodiments, a composition comprises 1 to 100 micromoles of a vehicle.

In some embodiments, a vehicle consists of one or more molecules that each have a boiling point between 40 and 260 degrees Celsius. In some specific embodiments, a vehicle comprises one or both of water and ethanol. In some specific embodiments, a vehicle comprises one or more terpenes. In some very specific embodiments, a vehicle comprises one or more terpenes selected from alpha-bisabolol, alpha-pinene, beta-caryophyllene, beta-pinene, humulene, limonene, linalool, myrcene, nerolidol, terpineol, and terpinolene.

In some embodiments, a composition is a gas phase composition comprising a pharmaceutical agent and a vehicle; the gas phase composition has a total pressure; the pharmaceutical agent has a first partial pressure; the vehicle has a second partial pressure; the first partial pressure is 0.1% to 50% of the total pressure; and the second partial pressure is 50% to 99.9% of the total pressure. In some specific embodiments, a composition is a gas phase composition comprising a pharmaceutical agent and a vehicle; the gas phase composition has a total pressure; the pharmaceutical agent has a first partial pressure; the vehicle has a second partial pressure; the first partial pressure is 1% to 40% of the total pressure; and the second partial pressure is 60% to 99% of the total pressure.

What is claimed is:

1. A container, comprising (i) a hermetically-sealed chamber, (ii) a composition contained within the hermetically-sealed chamber, and (iii) a thermally-conductive surface in thermal communication with the composition, wherein:
the composition comprises 0.01 to 500 micromoles of a pharmaceutical agent;
the pharmaceutical agent has a boiling point between 26 and 260 degrees Celsius; and
the thermally-conductive surface has a thermal conductivity greater than 2 watts per meter-Kelvin (W·m⁻¹·K⁻¹).

2. The container of claim 1, wherein the hermetically-sealed chamber contains less than 0.1 milligrams of molecular oxygen (O₂).

3. The container of claim 1, wherein the thermally-conductive surface has a surface area of less than 0.01 meters squared.

4. The container of claim 1, comprising metal, wherein the thermally-conductive surface comprises the metal, and the metal has a thermal conductivity of at least 2 watts per meter-Kelvin.

5. The container of claim 1, comprising an exit path, wherein the container is configured to inhibit the flow of the composition through the exit path until after the pharmaceutical agent is heated to a temperature above the boiling point of the pharmaceutical agent.

6. The container of claim 1, wherein the hermetically-sealed chamber is configured such that the composition can exist in either a liquid phase or a gas phase within the hermetically-sealed chamber; the hermetically-sealed chamber has an initial volume when the composition is in the liquid phase; the hermetically-sealed chamber has an inflated volume when the composition is in the gas phase; and the inflated volume is at least 900% greater than the initial volume.

7. The container of claim 6, wherein the initial volume is 0.01 to 100 microliters, and the inflated volume is 0.01 to 100 milliliters.

8. The container of claim 1, wherein: the composition comprises 1 to 1000 micromoles of a vehicle; the vehicle consists of one or more molecules that each have a boiling point between 40 and 260 degrees Celsius; and each molecule of the vehicle has a different molecular formula than the pharmaceutical agent.

9. The container of claim 8, wherein the vehicle comprises one or both of water and ethanol.

10. The container of claim 1, wherein the composition has a volume of 0.5 to 50 microliters.

11. The container of claim 1, wherein: the hermetically-sealed chamber comprises a first surface and a second surface; the composition is in physical communication with both the first surface and the second surface such that the composition separates at least a portion of the first surface from at least a portion of the second surface; and the average distance between the portion of the first surface and the portion of the second surface that are separated by the composition is 20 nanometers to 2 millimeters.

12. The container of claim 1, wherein: the composition is a gas phase composition; the gas phase composition has a total pressure; the pharmaceutical agent has a first partial pressure in the gas phase composition; the vehicle has a second partial pressure in the gas phase composition; the first partial pressure is 0.1% to 50% of the total pressure; and the second partial pressure is 50% to 99.9% of the total pressure.

13. A device, comprising a heating compartment and a heating element, wherein:
the heating compartment is configured to receive a container that comprises (i) a hermetically-sealed chamber, (ii) a composition that is contained within the hermetically-sealed chamber and that comprises a pharmaceutical agent, and (iii) a thermally-conductive surface in thermal communication with the composition; and
the heating element is configured to be in thermal communication with the thermally-conductive surface after the heating compartment receives the container such that the heating element is operable to (i) heat the thermally-conductive surface and (ii) heat the composition to (i) vaporize at least a portion of the pharmaceutical agent and (ii) produce a vaporized pharmaceutical agent.

14. The device of claim 13, comprising a controller in directive communication with the heating element, wherein:
the controller is configured to receive one or more inputs;
the controller is configured to run an authorization program comprising one or more boolean functions that determine whether the controller is authorized to run a heating program based on the one or more inputs;
the controller is configured to run the heating program when the controller is authorized to run the heating program; and
running the heating program causes the heating element to heat the thermally-conductive surface of a container contained in the heating compartment of the device.

15. The device of claim 14, wherein the one or more inputs comprise variable data and authorization data; the variable data comprises one or more of (i) the present date, (ii) the present time, (iii) the present location of the device, (iv) the present velocity of the device, (v) an identifier associated with a person who is operating the device that identifies the person; and the authorization data comprises one or more of (i) a date, a plurality of dates, or a range of dates during which the device may administer the pharmaceutical agent of the composition of the container contained within the heating compartment of the device; (ii) a time, a plurality of times, or a range of times during which the device may administer the pharmaceutical agent; (iii) a location, a plurality of locations, or a range of locations at which the device may administer the pharmaceutical agent; (iv) a velocity, a plurality of velocities, or a range of velocities at which the device may administer the pharmaceutical agent; and (v) an identifier that is associated with an authorized person who is authorized to either operate the device, administer the pharmaceutical agent, or both operate the device and administer the pharmaceutical agent.

16. The device of claim 15, wherein either (i) the identifier associated with the person who is operating the device is a password, an electromagnetic signal, or biometric data, or (ii) the identifier is either directly related to or dependent upon a password, an electromagnetic signal, or biometric data.

17. A method to prepare a vaporized pharmaceutical agent, comprising:
providing a container that comprises (i) a hermetically-sealed chamber, (ii) a composition that is contained within the hermetically-sealed chamber and that comprises a pharmaceutical agent, and (iii) a thermally-conductive surface in thermal communication with the composition;
heating the thermally-conductive surface to heat the composition and to vaporize at least a portion of the pharmaceutical agent to produce a gas phase composition comprising a vaporized pharmaceutical agent;
connecting the hermetically-sealed chamber of the container to an administration path to convert the hermetically-sealed chamber into an unsealed chamber such that the gas phase composition is in fluid communication with the administration path; and
directing the gas phase composition out of the unsealed chamber and into the administration path.

18. The method of claim 17, wherein producing the gas phase composition inflates the hermetically-sealed chamber from an initial volume to an inflated volume; and the inflated volume is at least 500% greater than the initial volume.

19. The method of claim 17, wherein the initial volume is 0.01 to 250 microliters and the inflated volume is greater than 0.5 milliliters.

20. The method of claim 17, comprising deflating the unsealed chamber from the inflated volume to a deflated volume, wherein the inflated volume is at least 1000% greater than the deflated volume, and deflating the unsealed chamber directs the gas phase composition out of the unsealed chamber and into the administration path.
